(19) United States
(12) Patent Application Publication
(10) Pub. No.: US 2010/0172919 A1
(43) Pub. Date: Jul. 8, 2010

(54) NOVELTREATMENT FOR NEUROLOGICAL DISORDERS

(76) Inventors: Jan Grimm, Dubendorf (CH); Roger Nitsch, Zumikon (CH); Feng Chen, Zurich (CH)

Correspondence Address: LATIMER INTELLECTUAL PROPERTY LAW, LLP, P.O. BOX 711200, HERNDON, VA, 20171, US

(21) Appl. No.: 12/664,540

(22) PCT Filed: Jun. 16, 2008
(86) PCT No.: PCT/EP08/04834

§ 371 Date: Mar. 9, 2010

Related U.S. Application Data
(60) Provisional application No. 60/934,645, filed on Jun. 15, 2007.
(60) Provisional application No. 61/066,255, filed on Feb. 19, 2008.

(30) Foreign Application Priority Data
Jun. 15, 2007 (EP) 07 011 780.9

(51) Int. Cl. A61K 38/18; C07K 14/505; A61K 39/395; A61P 25/28; G01N 33/68

(52) U.S. Cl. 424/172.1; 530/380; 514/8; 436/86

(57) ABSTRACT

Provided are novel drugs and methods in the treatment as well as diagnosis of neurological disorders such as Alzheimer's disease and amyloid-beta pathology/amyloidosis. More specifically, the use of erythropoietin and analogs thereof for the treatment of Aβ peptide related brain impairments is described. Furthermore, the use of claudin-5 and variants thereof as biomarker for Alzheimer's disease and for the progression of Alzheimer's disease, respectively, is provided.

### FIELD OF THE INVENTION

The present invention relates to the technical field of neurological disorders and methods for the treatment of the same. More specifically, the present invention pertains to the treatment of disorders associated with the amyloidogenic processing of amyloid precursor protein (APP) and the amyloid beta (Aβ) peptide in particular. Furthermore, the present invention relates to the use of claudin-5 and variants thereof as biomarker for Alzheimer's disease as well as biomarker for the progression of Alzheimer's disease.

### BACKGROUND OF THE INVENTION

For a variety of serious neurodegenerative diseases, there exist no effective therapies or cures. For example, in Alzheimer's disease, the most common neurodegenerative disease and most frequent cause of dementia, progressive failure of memory and degeneration of temporal and parietal association-cortex result in speech impairment and loss of coordination, and, in some cases, emotional disturbance. Alzheimer's disease generally progresses over many years, with patients gradually-becoming immobile, emaciated and susceptible to pneumonia. According to the amyloid cascade hypothesis, accumulation of amyloid beta peptide (Aβ) plays a central role in AD (Hardy and Selkoe, 2002). Two types of Aβ pathology are present in the AD brain: the neuritic plaques, the Aβ deposits in the grey matter, and the cerebral amyloid angiopathy (CAA), the Aβ deposits in cerebral and meningeal vessels. Abnormal accumulation of Aβ is believed to cause formation of neurofibrillary tangles, synaptic and neuronal loss, resulting in functional brain disruption. Therefore, Aβ-related interventions are currently the focus for developing AD therapies.

### SUMMARY OF THE INVENTION

The present invention relates to the use of erythropoietin (EPO) and erythropoietin-like agents in the treatment, amelioration and prevention, respectively, of neurological disorders, in particular disorders associated with Alzheimer's disease or related diseases with amyloid beta (Aβ) pathology and amyloidosis. In particular, the present invention makes use of the surprising finding that systemically administered EPO can ameliorate early Aβ pathology and microvessel disintegrity in transgenic mice with AD-like amyloid pathology of neuritic plaques and CAA, and which develop behavioral deficits at young age. Thus, the present invention for the first time provides a medicament comprising EPO as the therapeutically effective ingredient for the treatment of Alzheimer's disease which is indicated by amyloidogenic processing of APP and presence of Aβ in the brain, respectively, and microvessel disintegrity characterized by cell membrane disassociation of claudin-5 and its reduced protein level. In this context, the present invention also pertains to a method for assessing the presence and status, respectively, of Alzheimer's disease comprising measuring in a sample the level of caudin-5 or a variant thereof, preferably an about 16 kDa species, wherein a decreased level of claudin-5 and/or increased level of said variant thereof as compared to a reference value of a sample from a healthy subject is indicative that said individual suffers from or is at risk to suffer from Alzheimer's Disease.

Other embodiments of the invention will be apparent from the description that follows.

### BRIEF DESCRIPTION OF THE DRAWINGS

FIG. 1: rHuEPO ameliorates Aβ pathology. Thioflavin-S staining revealed compact Aβ plaques in the cortex of tg ctr (A). The number of cortical thioflavin-S plaques was significantly reduced in EpoL (B) and EpoH (C). Scale bar, 100 μm. (D) When compared with tg ctr, the average number of plaques was reduced by more than 40% in EpoL and EpoH (*P<0.05, **P<0.01, LSD).

FIG. 2: rHuEPO modulates astrocytes activity in response to Aβ deposits. Double immunofluorescent staining with 6E10 (red) and anti-GFAP antibody (green) on sagittal brain sections revealed strong astrocytosis associated Aβ plaques in the cortex of tg ctr (A and D). Both EpoL (B and E) and EpoH (C and F) had significantly less number of Aβ plaques and weaker astrocytosis. Parenchymal Aβ plaque-associated astrocytosis was markedly reduced in EpoL (H) and EpoH (I) compared with tg ctr (G). Scale bars, 100 μm.

FIG. 3: rHuEPO ameliorates CAA. At the age of eight months, arc Aβ mice already developed pronounce CAA both in leptomeningeal and parenchymal blood vessels (A and B). In tg ctr (), the cortical thioflavin-S plaque load was positively associated with the appearance of thioflavin-S positive blood vessels (r=0.725, P<0.05), but not in EpoL (□) (P=0.253) nor in EpoH (Δ) (P=0.647, Spearman's rho correlation coefficient test)(C). At normal conditions, astrocytes closely associate with brain blood vessels. In tg ctr, when blood vessels were heavily laden with Aβ, astrocytes detached from the vessel wall (D). In contrast, in EpoL and EpoH, astrocyte endfeet still closely enveloped the blood vessel wall that was laden with Aβ (E and F). Scale bars, 100 μm.

FIG. 4: rHuEPO reduces Aβ in the brain and serum. ELISAs that are specific for Aβ40 and Aβ42 were used. (A) Brain Aβ40 in RIPA fraction only slightly increased in tg ctr (n=9) compared with four-month-old arc Aβ mice (tg young, n=3), whereas, Aβ40 in SDS and FA fraction increased by four- and 40-fold respectively. rHuEPO lowered brain Aβ40 levels in SDS and FA fraction by more than 40% in EpoL (n=11) and EpoH (n=12). (B) rHuEPO also reduced brain Aβ42 levels in RIPA and FA fractions by more than 40%. (C) Pearson correlation analysis indicates strong associations between cortical thioflavin-S plaque load and brain Aβ levels (SDS and FA fraction for Aβ40 and RIPA and FA fraction for Aβ42; P<0.001), of which the association between the cortical plaque load and brain Aβ40 in SDS fraction (r=0.791) was the strongest, (□) for EpoH, (+) for EpoL and (Δ) for tg ctr. (D) rHuEPO significantly reduced the levels of Aβ40 in the serum of treated mice. Compared with tg ctr, P#<0.01 by 2-tailed student t-test, and P*=0.055, **<0.05, ***<0.01 by LSD.

FIG. 5: rHuEPO promotes non-amyloidogenic processing of APP. Antibody against C-terminal APP was used for Western blotting (A and B), which recognize full length APP (FL-APP) and C-terminal fragments of β-cleavage (β-CTF) and α-cleavage (α-CTF). In wt, only α-cleavage was detectable, whereas, β-cleavage was predominant in arcAβ mice (tg ctr and EpoH). The level of β-CTF was apparently higher in tg ctr than EpoH. The ratio of β-CTF to FL-APP based on densitometry measurements, was significantly reduced by 34% in EpoH compared with that of tg ctr (n=7, p*<0.01, student t-test).

These results are confirmed with SweAPP293 cells overexpressing human APP695 containing the Swedish mutation which were subjected to rHuEpo. The levels of α-CTF and β-CTF in SweAPP cells were approximately equal on Western blot (C). rHuEPO at various concentrations markedly increased α-CTF/β-CTF, which peaked at 1 UI/ml with α-CTF/β-CTF at 2.6. However, DAPT, an established γ-cleavage inhibitor, failed to block the increase in α-CTF/β-CTF by rHuEPO (D). In addition, the extracellular fragment of α-cleavage, sAPPα, was also increased in the conditioned media from rHuEPO treated cells (E). Thus, rHuEPO induced nonamyloidogenic processing of APP in arcAβ mice and in SweAPP293 cells.

FIG. 6: rHuEPO prevents Aβ toxicity on microvessel endothelial cells. Immunofluorescence staining of isolated brain microvessels showed an evenly distributed claudin-5 in wt (A), but a dotted distribution or completely loss of claudin-5 in Aβ-laden vessels in tg ctr (B). The disruption of claudin-5 distribution was less severe in rHuEPO treated mice (C). Red is for claudin-5, green for 6E10, and blue for DAPI. In contrast, Aβ deposition did not affect CD31 expression in microvessel endothelial cell (D, red for 6E10, green for CD31 and blue for DAPI). Astrocytes completely enveloped healthy brain microvessels, indicated by the even distribution of claudin-5 along the vessel wall in wt (E, red for claudin-5 and green for GFAP). In tg ctr, the lack of contact with astrocytes was in parallel to the loss of claudin-5 staining in the microvessels (F, red for claudin-5 and green for GFAP). Scale bar 20 μm.

FIG. 7: rHuEPO prevents loss of membrane claudin-5 induced by Aβ in endothelial cells. Endothelial cell line bEnd 5 was established from mouse brain microvessel. (A) After two weeks in culture, they expressed high amount of claudin-5 (red) in the cell membrane, which was co-localized with CD31 (green, blue for DAPI). However, after 24 hrs growing in 10 μM freshly prepared Aβ42, bEnd5 cells completely lost the cell membrane claudin-5; CD31 was still expressed in cell membrane while claudin-5 became accumulated in the cytoplasm. When 1 UI/ml rHuEPO was added together with 10 μM Aβ42, the cell membrane expression of claudin-5 was preserved. However, 1 UI/ml rHuEPO alone did not change the cell membrane distribution of claudin-5 nor CD31. Scale bar 20 μm. (B) Western blot reveals no difference in claudin-5 levels among control, 10 μM Aβ42, 1 UI/ml rHuEPO, and the combination of both treated cells. However, smaller C-terminal fragments appeared and increased in 10 μM Aβ42 treated cells. Treatment of 1 UI/ml rHuEPO alone had no effects on these C-terminal fragments, but significantly reduced the amount of small C-terminal fragment induced by Aβ.

FIG. 8: Western blot reveals the level of claudin-5 in the temporal cortexes from demented patients and healthy controls. Subjects only with a clinical diagnose of dementia were labeled as +, clinically non-demented subjects as −. The severity of neurofibrillary tangles was indicated by Braak and Braak Stage (B&B stage). In addition, the apolipoprotein E genotype of each subject was also indicated.

### DEFINITIONS

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2.

“Agent”, “reagent”, or “compound”, as the terms are used herein, generally refer to any substance, chemical, composition, or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or inorganic molecules or compositions, small molecules, drugs and any combinations of any of said agents above. They may be used for testing, for diagnostic or for therapeutic purposes.

If not stated otherwise, the terms “compound”, “substance” and “(chemical) composition” are used interchangeably herein and include but are not limited to therapeutic agents (or potential therapeutic agents), food additives and nutraceuticals. They can also be animal therapeutics or potential animal therapeutics.

“Small organic molecule”, as the term is used herein, refers to an organic compound [or organic compound complexed with an inorganic compound (e.g., metal)] that has a molecular weight of less than 3 kilodaltons, preferably less than 1.5 kilodaltons. Furthermore, the term “synthetic organic molecule” may be used interchangeably with the term “small organic molecule” except that the synthetic organic molecule is made by man and not to be found in nature unless stated otherwise.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Furthermore, the term “subject” as employed herein relates to animals in need of therapy, e.g. amelioration, treatment and/or prevention of neurological disorders such as Alzheimer's disease. Most preferably, said subject is a human.

### GENERAL TECHNIQUES

For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech.

### DETAILED DESCRIPTION OF THE INVENTION

The present invention is directed to the surprising discovery that erythropoietin (EPO) is capable of ameliorating brain parenchymal and vascular amyloid pathology, reducing Aβ levels in the brain and in serum and reversing behavioral abnormalities in a transgenic Alzheimer's disease mouse model, indicating that EPO can be useful in preventing and treating acute and chronic neurological disorders and amyloidogenic diseases such as, without limitation, Alzheimer's disease, Down's syndrome, amyotrophic lateral sclerosis (ALS), Huntington's disease, glaucoma, HIV-associated dementia, multiple sclerosis, Parkinson's disease, neuropathic pain, inclusion body myositis, and particularly sporadic and familial forms of cerebral amyloid angiopathy (CAA).

Erythropoietin (EPO) ameliorates brain damage caused by ischemia and inflammatory diseases besides its clinical use for treating anemia. However, a role for EPO in Alzheimer's disease (AD) is yet unknown. As disclosed herein in the Examples, EPO is capable of preventing or reversing the AD like pathology in APP transgenic mice when administered systemically. In particular, in order to examine whether EPO has a protective role in Alzheimer's disease (AD), three-month-old arcAβ mice as an AD mouse model, which developed Aβ plaques and cerebral amyloid angiopathy (CAA) at young age, were treated weekly with 18 UI or 1.8 UI (equivalent to 60 or 600 UI/kg, respectively) recombinant human EPO (rHuEPO) via intraperitoneal injection for five month.

As demonstrated in the Examples, brain Aβ plaque load was significantly reduced by more than 40% in rHuEPO treated arcAβ mice. These mice also had less severe CAA and astrocytosis associated with Aβ plaque. Consistently, ELISAs showed significant reduction in brain Aβ levels of RIPA-insoluble fractions as well as in serum Aβ40 levels. Furthermore, the ratio of C-terminal fragment of β-cleavage to full length APP was significantly reduced in rHuEPO treated mice, suggesting a shift of APP processing towards non-amyloidogenic pathway by rHuEPO. In addition to CAA, arcAβ mice had also compromised tight junction in brain microvessel endothelial cells, which was characterized by the disruption of paracellular distribution and reduced protein level of claudin-5. rHuEPO partially preserved the normal claudin-5 distribution. Prevention of Aβ toxicity on claudin-5 by rHuEPO was further confirmed in endothelial cell line obtained from mouse brain microvessel. Together, the present study demonstrates that rHuEPO reduced brain Aβ levels and prevented microvessel damage. It suggests rHuEPO as a potential treatment for AD.

As further investigated, rHuEPO treatment can ameliorate behaviour deficits in the APP transgenic mouse model. In addition, the treatment did not result in increased erythropoiesis and no EPO treatment-related adverse events were observed as disclosed herein in Example 6. Together, the present study suggests a beneficial role for rHuEPO towards the amelioration of Aβ-related pathology and behavioral abnormalities.

Based on these discoveries, the present invention provides a method of treating, ameliorating and preventing neurological disorders in a subject by inducing the erythropoietin (EPO) pathway. Accordingly, the present invention relates to erythropoietin (EPO) and active fragments and analogs thereof for the treatment, amelioration or prevention of a neurological disorder and/or amyloidosis, in particular a disorder associated with Alzheimer's disease or amyloid β (Aβ) pathology and/or amyloidosis.

Erythropoietin (EPO) is a type I cytokine which is mainly produced in the kidney of adult mammals in response to hypoxia. Recombinant human EPO (rHuEPO) is widely used to improve the life quality of patients with anemia. A wide range of non-erythroid cells, including astorcytes, neurons and brain capillary endothelial cells express abundant amount of functional receptor for EPO (EpoR) (Yamaji et al., 1996). EPO produced by astrocytes in response to various brain damages is crucial for the survival of affected neurons (Chong et al., 2005; Nadam et al., 2007). Numerous studies have shown that EPO is neuroprotective, as well as effective in promoting neurogenesis, synaptic plasticity and angiogenesis (Buemi et al., 2002; Brines and Cerami, 2005; Lu et al., 2005; Carmichael, 2006; Tsai et al., 2006). In addition, systematic rHuEPO can penetrate the blood brain barrier (BBB) (Brines et al., 2000) and is well tolerated in stroke patients (Ehrenreich et al., 2002). Therefore, rHuEPO may be a potential drug for diseases of the central nervous system (CNS). However, a role for rHuEPO in Alzheimer's disease (AD) has not been demonstrated so far.

Of course, in view of the being one of the blockbuster biopharmaceuticals, EPO has been claimed for use in nearly any treatment of every kind of disease or disorder including neurological disorders with Alzheimer's disease being a most prominent one; see for example international application WO2007/060213, the disclosure content of which is incorporated herein by reference for the purpose of supplementing the description of the present application with respect to possible EPO polypeptides described therein that may be useful in accordance with the teaching of the present invention.

Even if considered for the treatment of a neurological disorder, EPO has not been shown to be involved in or to be able to ameliorate any of the mechanisms underlying a neurological or neurodegenerative disorder such as Alzheimer's disease or Aβ pathology, let alone be proved to indeed be useful for the treatment of such a disorder in kind. However, since neurodegenerative disorders are quite complex and the result of alternative and/or cumulative causes and risk factors, it is essential to know, which and preferably how a proposed neurological drug targets the pathological pathway. This is particularly true for Alzheimer's disease and amyloidogenic disorders.

In context with Alzheimer's disease, previously EPO has been suggested in Japanese patent application JP5092928 for raising the intracellular calcium level of neurons and enhancing choline acetyltransferase activity in order to treat Alzheimer type dysmnesia, in particular via direct infusion into intracranial septal areas. As evident, this is not an embodiment of the present invention and any embodiment disclosed in JP5092928 that may be considered to fall within the ambit of the appended claims is disclaimed herewith.

In addition, EPO is almost always among so called washing lists of cellular growth factors and cytokines cited in patent applications to supplement a proposed therapeutically active agent when formulated in a pharmaceutical composition; see, e.g., international applications WO2005/028511 and WO2006/039470. These particular “combination preparations” are disclaimed herewith if considered to fall within the ambit of the appended claims. Hence, as evident from the appended examples EPO may preferably be the sole therapeutic agent for the treatment of Alzheimer's disease and Aβ pathology, respectively.

In accordance with the present invention it believed that the amyloid cascade, i.e. accumulation of amyloid beta peptide (Aβ) plays a central role in AD (Hardy and Selkoe, 2002), and consequently causes synaptic and neuronal loss, neurofibrillary tangle formation and brain malfunction. Two types of Aβ pathology are present in AD brain: neuritic plaques, the Aβ deposits in the grey matter, and cerebral amyloid angiopathy (CAA), the Aβ deposits in cerebral and meningeal vessels. Using transgenic mice that mimic the amyloid pathology of neuritic plaques and CAA in AD (Knobloch, 2006) it could be shown in accordance with the present invention for the first time that systemically administered rHuEPO can reduce brain Aβ levels and ameliorate Aβ-related brain microvessel damage.

Thus, the present invention for the first time provides EPO in a pharmaceutical composition for the therapeutic intervention in the treatment of Alzheimer' disease and other neurological disorders that involve Aβ pathology and brain microvessel damage, respectively.

The pharmaceutical compositions of the present invention can be formulated according to methods well known in the art; see for example Remington: The Science and Practice of Pharmacy (2000) by the University of Sciences in Philadelphia, ISBN 0-683-306472. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intra-muscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 mg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as dopamine or psychopharmacologic drugs, depending on the intended use of the pharmaceutical composition. Furthermore, the pharmaceutical composition may also be formulated as a vaccine, for example, if the pharmaceutical composition of the invention comprises an anti-Aβ antibody for passive immunization.

In addition, co-administration or sequential administration of other agents may be desirable. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The pharmaceutical compositions in accordance with the present invention can be used for the treatment of neurological disorders and/or amyloidosis including but not limited to Alzheimer's disease, cerebral amyloid angiopathy (CAA), Down's syndrome, mild cognitive impairment, hereditary cerebral hemorrhage with amyloidosis Dutch type and Icelandic type, Dementia with Lewy Bodies, vascular dementia, progressive supranuclear palsy, multiple system atrophy, corticobasal degeneration, frontotemporal degeneration with Parkinsonism liked to chromosome 17, frontotemporal dementia, aphasia, Bell's Palsy, Creutzfeldt-Jakob disease, epilepsy, encephalitis, Huntington's disease, neuromuscular disorders, neuro-oncology, neuro-immunology, neuro-otology pain, pediatric neurology, phobia sleep disorders, Tourette Syndrome, amyotrophic lateral sclerosis (ALS), inclusion body myositis, multiple sclerosis, HIV-associated dementia, HIV-associated neuropathy, neuropathic pain, migraine, glaucoma, drug addiction, drug withdrawal, drug dependency, depression, anxiety, Parkinson's disease, other movement disorders or diseases of the central nervous system (CNS) in general.

Importantly, the data obtained in accordance with the present invention suggest a direct involvement of rHuEPO in the development of Aβ pathology. The present data show that chronic rHuEPO treatment reduced brain Aβ plaque load and serum Aβ levels at the early stage of Aβ pathology in AD mice. Although a direct involvement of rHuEPO in APP processing may not be ruled out, without intending to be bound by theory it is believed that in accordance with the present invention EPO is directly involved in Aβ clearance and that the primary target of EPO in the brain was capillary endothelial cells, because EPO was systemically administered, and EpoR in endothelial cells has an affinity for EPO ten times higher than those in neurons and astroctyes (Brines and Cerami, 2005). Upon rHuEPO stimulation, endothelial cells proliferate, produce and secrete matrix metalloproteinase-2 (MMP-2) and MMP-9 (Ribatti et al., 1999; Wang et al., 2006a). MMP-2 and MMP-9 have been suggested as Aβ-degrading enzymes (Roher et al., 1994; Backstrom et al., 1996). Indeed, the enzymatic activity of MMP-9 has been shown to be lower in AD (Backstrom et al., 1996; Thirumangalakudi et al., 2006). This notion is supported by a recent study, showing that MMP-9 was able to degrade Aβ fibrils as well as compact Aβ plaques in vivo (Yan et al., 2006). In accordance with the present invention, it could be shown that rHuEPO treated mice had less severe CAA. Therefore, it is prudent to assume that enhanced production and activity of MMP-9 and MMP-2 in capillary endothelial cells may contribute to the decreased levels of β-amyloid deposits in blood vessels.

Astrocytes and microglia are another possible target of rHuEPO. For example, astrocytes express both EPO and EpoR, and are closely related to brain capillaries (Brines et al., 2004). Under normal physiological conditions, brain microvessels are completely enveloped by astrocytes, which play an important role in maintaining the BBB (Willis et al., 2004b). On the other hand, proinflammatory cytokines secreted by activated astrocytes, such as IL-6 and monocyte chemoattractant protein 1, can cause the BBB break down. In accordance with the present invention detachment of astrocytes from Aβ-laden blood vessels was observed in arcAβ mice. This could be caused either by Aβ toxicity directly on astrocyte endfeet or by the BBB leakage induced by Aβ. The experiments performed and described in the examples suggest that the BBB leakage caused by Aβ accumulation is at least in part, due to the dislocation of claudin-5. This is where rHuEPO might play a role, as could be shown here that rHuEPO prevented Aβ toxicity on claudin-5. Consequently, the close contact between astrocytes and microvessels was preserved by rHuEPO. Additionally, rHuEPO is known to promote astrocytes proliferation and inhibits the secretion of proinflammatory cytokines induced by Aβ (Villa et al., 2003). Together, the findings of the present invention strongly support a beneficial role of rHuEPO in maintaining normal microvesssel function upon Aβ insult.

Therefore, in accordance with the present invention it is proposed but without intending to be bound by that theory that EPO may function as a modulator for neurovascular units, which consist of microvessels, astrocytes and neurons. When applied properly, EPO may thus be a potential therapeutic approach for AD as well as other neurological disorders or amyloidoses.

Taken together with the results described in the examples below, it is envisaged that EPO does not have to be necessarily supplied exogenously, for example via systemic administration as performed in accordance with the examples but could also be expressed in a target cell, in particular endothelial cell and/or astrocyte or other cell which is associated with the mentioned neurovascular unit. Accordingly, in two alternative embodiments of the therapeutic use in accordance with the present invention, EPO or an active fragment or analog thereof is designed to be applied exogenously to or expressed in a target cell, preferably wherein said target cell is a capillary endothelial cell or astrocyte in the brain.

Many forms of erythropoietin, as well as active fragments and analogs thereof, can be useful in the methods of the invention. In one embodiment, said EPO or an active fragment thereof is human EPO or an active fragment thereof. In another embodiment, an EPO analog may be used, which can be, without limitation, a peptide, peptidomimetic, small molecule or nucleic acid EPO analog. In a further embodiment, the present invention is practiced with EPO, or an active fragment or analog thereof, which has at least 10-fold higher affinity for the EPO receptor than native human EPO. In another embodiment, the present invention is practiced with EPO or an active fragment or analog thereof which is oligomeric, for example, dimeric.

In a still further embodiment, the present invention is practiced with EPO or an active fragment or analog thereof that has a half-life greater than the half-life of native human EPO. In an additional embodiment, the present invention is practiced with EPO or an active fragment or analog thereof that is hyper-glycosylated compared to native human EPO. In yet a further embodiment, the present invention is practiced with Darbepoietin. In any embodiment of the present invention, soluble EPO receptor optionally can be included, for example, to increase the half-life of EPO or an active fragment or analog thereof.

As used herein, the term “erythropoietin” is synonymous with “EPO” and means a polypeptide that has substantially the amino acid sequence of naturally occurring human EPO or a homolog thereof. EPOs useful in the present invention include human and other primate EPOs, mammalian EPOs such as bovine, porcine, murine and rat homologs and other vertebrate homologs such as Danio rerio homologs. Thus, the term EPO encompasses species homologs, alternatively spliced forms, isotype and glycosylation variants and precursors-of the mature human EPO sequence.

If not stated otherwise, the terms “human recombinant EPO” and “rHuEPO”, respectively, and “EPO” may be used interchangeably herein for the description of its therapeutic use in accordance with the present invention.

General information about EPO and EpoR can be retrieved from public databases such as UniProtKB/Swiss-Prot; see for example primary accession number P01588 and secondary accession numbers Q2M2L6, Q549U2, Q9UDZ0, Q9UEZ5 and Q9UHA0 for the amino acid sequence of EPO as well as the references cited therewith. The cloning of the human EPO gene and its recombinant expression has been described in European patent application EP 0 148 605 A2.

Erythropoietin (EPO), synonymous with epoetin, is the principal hormone involved in the regulation of erythrocyte differentiation and the maintenance of a physiological level of circulating erythrocyte mass. Originally, EPO has been described for use in the treatment of anemia. EPO preparations at a pharmaceutical grade are commercially available, for example under the names Epogen (Amgen), Epogin (Chugai), Epomax (Elanex), Eprex (Janssen-Cilag), NeoRecormon or Recormon (Roche), and Procrit (Ortho Biotech). Variations in the glycosylation pattern of EPO distinguishes these products. Epogen, Epogin, Eprex and Procrit are generically known as epoetin alfa, NeoRecormon and Recormon as epoetin beta and Epomax as epoetin omega.

Erythropoietin receptor (EPO-R, EpoR) is also well known to the person skilled in the art; see for example primary accession number P19235 as well as secondary accession numbers Q15443 and Q2M205 for the amino acid sequence of EPO-R as well as the references cited therewith. EPO-R is the receptor for EPO and mediates EPO-induced erythroblast proliferation and differentiation. Upon EPO stimulation, EPO-R dimerizes and triggers the JAK2/STAT5 signaling cascade. In some cell types, EPO-R can also activate STAT1 and STAT3 and may also activate the LYN tyrosine kinase.

EPO analogs that can be used in accordance with the present invention have also been described in the prior art. For example, Long et al., Exp. Hematol. 34 (2006), 697-704, describe the design of homogeneous, monopegylated EPO analogs with preserved in vitro bioactivity by targeted attachment of maleimide-PEGs to engineered EPO cysteine analogs. EPO fusion analogs such as human serum albumin (EPOa-hSA) fusion protein and human IgG (EPO-IgG) fusion protein as well as methods of making the same are described in international application WO99/66054 and WO2005/079232, respectively.

Hyperglycosylated EPO analogs and methods of their production are also described, for example in international application WO00/24893.

In addition, EPO analogs have been described, which act as agonists and effect dimerization of the EPO receptor and thus signal initiation; see, e.g., international application WO96/40772. A particle formulation including an erythropoietin receptor agonist, a buffer, and a sugar, wherein the buffer and sugar stabilize the erythropoietin receptor agonist against aggregation, is disclosed in international application WO2006/017773. The EpoR agonist described therein may be used in accordance with the present invention either alone or in the mentioned formulation.

Furthermore, EPO analogs of EPO have been described, which are not directly derived from EPO. For example, EPO analogs are available that do not bind to the dimeric EPO receptor and lack erythropoietic activity, e.g., carbamylated EPO (CEPO); see, e.g., Fiordaliso et al., Proc. Natl. Acad. Sci. USA 102 (2005), 2046-2051 and the references cited therein. In addition, the EPO receptor can be activated to signal cell growth by binding F-gp55, the Friend spleen focus-forming virus glycoprotein; see, e.g., Barber et al., Mol. Cell. Biol. 14 (1994), 2257-2265. Thus, an EPO fragment and analog, respectively, will usually have substantially the same activity on the EpoR as native EPO.

A detailed summary of different kinds of EPO and EpoR, active fragments and analogs thereof as well as nucleic acid based EPO application which are included in the term “EPO” for the purpose of the present invention is given in international application WO03/103608 at page 23, line 14 to page 34, line 26, the disclosure content of which is incorporated herein by reference.

However, if not already evident from the specified neurological condition, medical use, treatment regimen, dose and/or target cell in the claims and herein, for the sake of clarity it is to be understood that in most if not all embodiments the present invention does not encompass a method of providing acute neuroprotection by inducing an insulin-like growth factor (IGF) signaling pathway in the neuronal cells close to or subsequent to the time of excitatory insult, thereby producing a synergistic acute neuropotective effect in the neuronal cells as disclosed and taught in international application WO03/103608, which disclosure is explicitly excluded herewith from the scope of the claimed invention.

Hence, in accordance with the present invention the term “EPO” includes any kind of EPO, active fragment and analog thereof, respectively, in particular those which retain one or more of the biological activities discovered for EPO for the first time as described in the above and the examples herein unaffected in kind, i.e.
(a) reducing cortical Aβ plaque load;
(b) reducing soluble and insoluble brain Aβ level;
(c) reducing serum Aβ level;
(d) ameliorated the cerebral amyloid angiopathy (CAA);
(e) reducing Aβ plaque-associated astrocytosis;
(f) preventing amyloidogenic APP processing;
(g) substantially maintaining or restoring normal distribution of full-length claudin-5, and
(h) improving abnormal behaviour.

These novel biological activities of EPO can be tested in accordance with the present invention, for example in arcAβ mice (Knobloch et al., “Intracellular Abeta and cognitive deficits precede beta-amyloid deposition in transgenic arcAbeta mice” [epub ahead of print] Neurobiol Aging 2006 Jul. 28; S1558-1497) as described in the examples. On the other hand, a useful EPO analog in accordance with the present invention may be devoid of the effects of EPO, which are not required for the therapy of neurological disorders as disclosed herein, e.g., thrombogenesis.

As explained above and in the discussion of the examples, one theory underlying the present invention is that the Aβ lowering effect of EPO is due to a synergistic activation of astrocytes and capillary endothelial cells, which are modulated by EPO. Thus, an EPO analog in accordance with the present invention also can be a modulator, in particular activator/agonist of astrocytes and/or capillary endothelial cells. Activator and agonists of astrocytes and capillary endothelial cells are known in the art and further can be identified by routine methods; see, e.g., U.S. Pat. No. 5,728,534 and Selmaj et al., J. Immunol. 144 (1990), 129-135; the disclosure contents of which are incorporated herein by reference.

An important finding in accordance with the present invention is that rHuEPO significantly reduced brain Aβ levels in arcAβ mice, which was accompanied by the reduction of cerebral plaque load and CAA. With intending be bound by theory it is believed that the reduction of Aβ is due to the non-amyloidogenic processing of APP induced by rHuEPO. The arcAβ mice express human APP with both Swedish and Arctic mutation in the brain. This renders APP predominantly undergoing β-cleavage instead of α-cleavage, due to the high activity of BACE1 in the brain. Under this circumstance, less β-CTF means less substrate for the amyloidogenic processing of APP, and eventually less Aβ product. Indeed, a significant reduction of β-CTF was reduced in rHuEPO treated mice. In the present examples, the cell culture experiments showed that the ratio of α-CTF to β-CTF and the level of secreted sAPPα in culture medium were greatly increased upon rHuEPO treatment in swAPP293 cells. This further suggests that rHuEPO favors α-cleavage over β-cleavage. Following the line of β-cleavage of APP, it is prudent to assume that rHuEPO affects APP processing through its function of anti-hypoxia. Hypoperfusion is a common feature of AD, which causes poor oxygen and energy supply to the brain (Iadecola, 2004). Hypoxia regulates the expression of many genes through the hypoxia inducible factor (HIF-1), including BACE1, which plays a role in amyloidogenesis in APP transgenic mice (Sun et al., 2006). Therefore, while not confined to theory in accordance with the present invention it is believed that there exists a vicious cycle, such that the brain blood vasculature damage caused by Aβ accumulation results in hypoperfusion and hypoxia, which in turn induces BACE1 production and amyloidogenesis. EPO improves local cerebral blood flow impaired by ischemia (Li et al., 2007), and promotes angiogenesis (Jaquet et al., 2002), whereby improves the compromised oxygen and energy supply. By improving hypoxia condition, rHuEPO might be able to negatively regulate hypoxia-induced BACE1, whereby reduce Aβ production. The reduction in serum Aβ levels in rHuEPO treated mice also indirectly suggested a role of rHuEPO in regulating Aβ production.

Accordingly, in one particular embodiment the present invention relates to EPO for preventing amyloid precursor protein (APP) amyloidogenic processing and thus treatment of related disorders.

As disclosed herein in the examples, EPO or an active fragment or analog thereof can be conveniently administered systemically, for example by intraperitoneal injection. However, other administration routes may be used as well such as those referred to above. Despite reported beneficial effects for brain damages, potential adverse effects of rHuEPO should however not be neglected.

Excessive erythropoiesis causes impaired learning (Rifkind et al., 1999) and shortened life span (Ogunshola et al., 2006). It is known that erythropoiesis in response to rHuEPO is dose and administration frequency dependent. Weekly ip injection of 5000 UI/kg rHuEPO did not significantly increase the hematocrit in mice, whereas three times a week over 5 weeks ip injection of 125 UI/kg rHuEPO only generated a modest increase in hematocrit (Egrie et al., 2003). Therefore, proper dosing is crucial to avoid these adverse effects. In accordance with the present invention it was found that arcAβ mice received weekly 600 UI/kg or 60 UI/kg rHuEPO had a similar hematocrit to that of wild type controls, although they had slightly higher level of hemoglobin than saline treated arcAβ mice. Thus, it could be shown that rHuEPO ameliorated Aβ pathology and microvessel disintegrity without inducing excessive erythropoiesis in mice. As allometric scaling from mouse to human is often scaled by a factor of 10, it is prudent to assume that the effective dose in human would be below the effective dose determined in mice.

Thus, one particular advantage of the therapeutic use of EPO or active fragment or analog thereof in the treatment of for example Alzheimer's disease is that it can be administered at a therapeutically effective dose which does not lead to a significant increase of the hemoglobin level and the hematocrit of the subject to be treated or at least only to an extent which is acceptable compared to the therapeutic effect for the subject. Thus, the present invention can be preferably practiced by administering a dose of at most 1000 U/kg EPO, or active fragment or analog thereof. In particular embodiments, the present invention is practiced by administering a dose of at most 750 U/kg, 500 U/kg, 250 U/kg, 100 U/kg, 90 U/kg, 80 U/kg, 70 U/kg, 60 U/kg, 50 U/kg, 40 U/kg, 30 U/kg, 20 U/kg, 10 U/kg, 5 U/kg, 2.5 U/kg or 1 U/kg EPO or active fragment or analog thereof. As explained above and in the examples, irrespective the single dose unit and administration regimen, respectively, EPO or an active fragment or analogue thereof can be administered at therapeutic doses which are lower than previously observed and have been calculated to be of at most 1000 UI/kg, or taken the conversion factor from mouse to human into account of at most 100 UI/kg, preferably at most 60 UI/kg and most preferably at most 10 UI/kg, in particular if the pharmaceutical composition comprising EPO or an active fragment or analogue thereof is administered weekly, which is one of the preferred administration regimen in accordance with the present invention. Thus, in one embodiment of the present invention, the pharmaceutical composition comprising EPO or an active fragment or analog thereof is designed to be administered at a dose of at most 1000 UI/kg, preferably of at most 500 UI/kg, more preferably of at most 100 UI/kg, most preferred of at most 50 UI/kg or less as mentioned before and on a weekly basis either as single or consecutive treatment.

As explained above and demonstrated in the examples, the methods of the present invention of preventing or reducing Aβ pathology in a subject are based, in part, on the discovery that EPO may be expected to be therapeutically active for this purpose at lower doses than previously observed for the treatment of other diseases. Thus, the present invention relates to a method for the treatment, amelioration or prevention of a neurological disorder in a subject, comprising administering to said subject EPO or an active fragment or analog thereof as defined above preferably at a dose of at most i.e. just even less than 1000 UI/kg, thereby preventing or reducing the severity of the neurological disorder or amyloidosis. However, as mentioned above, higher doses, i.e. 1250, 1500, 2000 UI/kg or even more may be applied as well if needed to achieve the therapeutic effect while the side effect of, e.g., erythopoiesis is still negligible or may be medically justifiable.

Furthermore, according to one non-binding theory underlying the present invention, EPO's useful therapeutic effect in the treatment of Alzheimer's disease, in particular as related to EPO's capability of lowering brain parenchymal and vascular amyloidosis as well as the levels of brain and serum Aβ is due to an activation of astrocytes and/or capillary endothelial cells. Thus, in one embodiment, the therapeutic use of EPO includes contacting astrocytes and capillary endothelial cells, respectively, or both with EPO or an active fragment or analog thereof, thereby inducing or enhancing the production and activity, respectively, of matrix metalloproteinase (MMP) in particular MMP-2 and/or MMP-9.

In one particular aspect, the present invention provides a method of ameliorating or treating a neurological disorder or amyloidosis in a subject by administering to the subject EPO or an active fragment or analog thereof in a therapeutically effective amount as defined above, thereby preventing or reducing brain Aβ plaque load or brain and serum Aβ level, respectively. In a particular preferred embodiment, the therapeutic use of EPO or an active fragment or analogue thereof is characterized by selectively reducing the vascular deposition of Aβ resulting in the treatment of cerebral amyloid angiopathy (CAA).

Since the therapeutic approach of the present invention for treating Alzheimer's disease and amyloidosis, respectively, does not rely on directly targeting APP processing and Aβ, advantageous and even synergistic effects may be expected when EPO and EPO-like agents are used in addition or combination with drugs commonly used in Aβ-related interventions such as those described in the prior art. Preferably, the additional drug is an anti-Aβ antibody or an equivalent binding molecule. Anti-Aβ antibodies and other Aβ binding molecules are well known in the prior art. Preferred human anti-Aβ antibodies and equivalent binding molecules are disclosed in applicant's co-pending international application, serial number PCT/EP2008/000053 “Method of providing disease-specific binding molecules and targets”, filed on Jan. 7, 2008 (attorney's docket: NE30A06/P-WO), the disclosure content of which is incorporated herein by reference. Of course, other drugs thought to be useful in the treatment of neurological disorders, in particular Alzheimer's disease can be used in combination with EPO and EPO-like molecules as well as, for example those described in Klafki et al., Brain 129 (2006), 2840-2855. Epub 2006, Oct. 3; Melinkova, Therapies for Alzheimer's disease, Nat. Rev. Drug Discov. 6 (2007), 341-342; Pipeline and Commercial Insight: Alzheimer's Disease Beta Treatments on the Horizon; A Datamonitor Report, published: November 05; Product Code: DMHC212.

Hence, in one embodiment the present invention relates to a drug combination preparation comprising EPO or an EPO-like molecule as described hereinbefore and an Aβ-specific drug, preferably an anti-Aβ antibody. Naturally, the combined drug preparation is especially useful for the treatment of the disorders described supra, in particular Alzheimer's disease and amyloidosis.

In a further embodiment, the therapeutic uses and methods of the present invention comprise administering the pharmaceutical composition including EPO or EPO-like molecules described hereinbefore in conjunction with a pharmaceutical composition including an Aβ-specific drug, preferably an anti-Aβ antibody or equivalent binding molecule. Administration of the two or more pharmaceutical compositions may be concurrently or subsequently in any way.

As demonstrated in the examples rHuEPO improved the brain microvessel integrity. Microvasculature damage causes the disruption of BBB. The tight junction formed between endothelial cells defines the BBB properties of low paracellular permeability and high electrical resistance. The thigh junction is composed of three major groups of proteins, occludin, junctional adhesion molecule-1 and the claudin family, of which claudin-5 is predominantly expressed in brain microvessels. Claudin-5 is negatively regulated by inflammatory changes (Gurney et al., 2006) and hypoxia (Koto et al., 2007). Phosphorylation of claudin-5 by protein kinase A is thought to be crucial for the barrier function (Soma et al., 2004). In mice, vasculature is not only closely related to Aβ plaques (Kumar-Singh et al., 2005), but also particularly vulnerable to Aβ (Park et al., 2004). Aβ42 fibrils induce the dislocation of claudin-5 from the plasma membrane to the cytoplasm in brain endothelial cells (Marco and Skaper, 2006). In accordance with the present invention it was further demonstrated that 10 μM of freshly prepared Aβ42 already induced the dislocation of claudin-5 from the cell membrane to the cytoplasm in bEnd5 cells. In addition, four C-terminal fragments with the size of between 6 to 16 kDa were observed on the Western blot in Aβ42 treated cells (FIG. 7B). The appearance of a substantial amount of C-terminal fragments indicated an abnormally high turn-over of claudin-5 and a destabilized tight junction induced by Aβ42. Indeed, the BBB permeability of fluorescein sodium salt (376 Da), was dramatically increased in arcAβ mice that had no detectable microhemorrhage. The present data as well as others (Willis et al., 2004a) suggest claudin-5 as an earlier marker for microvasculature damage and BBB leakage. Cerebral microvasculature damage in AD is highly prevalent; 80-90% of AD patients have CAA. Thus, it is prudent to expect an important role of claudin-5 in AD and strongly support the hypothesis that vasculature lesion is a strong factor for the disease.

More importantly, in accordance with the present invention it could be demonstrated that rHuEPO ameliorated Aβ-toxicity towards claudin-5 both in vitro and in vivo and thus seems to have a protective effect in microvessel endothelial cells from human brain. Hence, the normal distribution of claudin-5 was partially restored by rHuEPO. rHuEPO preventing Aβ toxicity on claudin-5 was further confirmed in murine brain microvessel endothelial cell line. Without intending to be bound by theory it is, due to the findings of the present invention, prudent to expect that EPO can be used to ameliorate microvessel disintegrity which is due to a disturbed claudin-mediated cell adhesion. Thus, the present invention also relates to a pharmaceutical composition comprising erythropoietin (EPO) or an active fragment or analog thereof for the treatment, amelioration or prevention of a claudin-mediated cell adhesion condition, in particular conditions which are associated with a neurological disorder. Hence, the present invention also encompasses a method for increasing tight junction formation activity or epithelial or endothelial barrier function activity in a subject in need thereof, comprising administering EPO. Within certain embodiments, EPO may be used to increase blood/brain barrier permeability and thus be administered with other drugs which are intended to exert their effects in the brain.

In a still further aspect, the present invention relates to a method for assessing Alzheimer's disease in vitro comprising measuring in a body fluid sample the level of caudin-5 or a variant thereof, wherein a decreased level of claudin-5 and/or increased level of an about 16 kDa variant thereof as compared to a reference value of sample from a healthy subject is indicative that said individual suffers from or is at risk to suffer from Alzheimer's disease. As demonstrated in the examples and shown in FIG. 8, in AD demented patients, the full length form of claudin-5 was absent while a smaller band of 16 kDa was detected as the dominant form. The difference in the level of full length claudin-5 and the appearance of low molecular weight forms between demented patients and healthy control subjects indicates that claudin-5 and its 16 kDa variant is a prominent marker of brain vasculature damage in Alzheimer's disease.

Thus, the present invention further relates to an in vitro method for monitoring the progression of the Alzheimer's disease comprising measuring in a body fluid sample the level of caudin-5 or a variant thereof, wherein a decreased level of claudin-5 and/or increased level of an about 16 kDa variant thereof, compared with an earlier measurement of the level of claudin-5 or variant thereof is indicative for the progression of Alzheimer's disease. The body fluid may be cerebrospinal fluid or blood.

General means and methods for measuring in a body fluid sample the level of one or more proteins or their encoding nucleic acids are well known to the person skilled in the art; see, e.g., the general textbooks and manuals referred to herein and in the examples. For example, international application WO2007/140971 describes methods for assessing Alzheimer's disease in vitro comprising measuring in a body fluid sample the level of myelin-associated glycoprotein precursor (MAG), contactin associated protein 1 precursor, myelin oligodendrocyte glycoprotein precursor I (MOG), and others, wherein an altered level of one of said proteins is indicative that said individual suffers from Alzheimer's disease. These methods may be applied and adapted in accordance with present invention for determining the level of claudin-5 or a variant thereof, the purpose for which the disclosure content of international application WO2007/140971 is incorporated herein by reference.

In addition, the present invention relates to a kit comprising a means or an agent for measuring claudin-5 or variant thereof such as antibody or nucleic acid probe for use in the above-mentioned method; see also the appended examples. The kit may further comprise a user's manual for interpreting the results of any measurement with respect to determining the risk of an individual suffering from Alzheimer's disease.

Hence, the present invention relates to pharmaceutical compositions, methods, uses and kits substantially as herein before described especially with reference to the following examples.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc.) are hereby expressly incorporated by reference; however, there is no admission that any document cited is indeed prior art as to the present invention. A more complete understanding can be obtained by reference to the following specific examples which are provided herein for purposes of illustration only and are not intended to limit the scope of the invention.

### EXAMPLES

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

### Supplementary Methods

### Transgenic Mice

arcAβ mice expressing human APP695 with the Swedish (K670N-M671L) and the Arctic (E693G) mutation under the control of murine Prp promoter were bred on C57BL/6 and DBA/2 mixed background. Genotype was determined by PCR of tail genomic DNA. Mice were kept on a 12 hour light/dark cycle at 22° C. Food pellets and water were available ad libitum. This animal research has been approved by the local animal studies committee.

### EPO Treatments

Three-month-old arcAβ mice were weekly intraperitoneally (ip) injected with rHuEPO (Eprex, Janssen-Cilag AG, Baar, Switzerland) with a dose of 1.8 UI/mouse (EpoL, n=11) or 18 UI/mouse (EpoH, n=12), or with saline (tg ctr, n=10). A group of wild type mice was also treated with saline (wt, n=13). All groups were age matched and gender balanced. Treatments had been terminated a week before mice were killed.

### Y-maze Behaviour Testing

After 3 month of treatment, mice were tested in the Y-maze behavioural paradigm (Wolfer et al., 2004; Knobloch et al., 2006). The animals were put on a reversed 12 hour light/dark cycle two weeks before the tests. The examiner was blind to the treatments throughout the testing period. The general health status of the mice was assessed with the mini-neurological examination (Knobloch et al., 2006).

### Tissue Preparation

Mice were transcardially perfused with 10 ml 50 mM TrisCl (pH 7.4) and 6 mM EGTA under deep anesthesia (1.25% ketamin and 0.25% xylazin, 10 μl/g bodyweight). Blood was collected and allowed to coagulate at room temperature for 40 min. Serum was then collected and stored at −80° C. after centrifugation at 2,000 g 4° C. for 10 min. Brains were removed, and the left hemispheres were snap frozen on dry ice, the right immersion-fixed in 4% paraformaldehyde in PBS (pH7.4) at 4° C. for 24 hrs. The fixed brains were then embedded in paraffin. Serial sagittal sections of 5 μm thick were collected with a microtome.

### Aβ Plaque Quantification

Compact Aβ plaques were determined by standard thioflavin-S staining and were counted in five serial sections which evenly covered the brain area between LAT 0 mm and LAT −1 mm.

### Hematocrit Measurement

Erythrocyte hematocrit of the tail blood collected at 10-11 am was measured with a microcapillary after centrifugation, quantified by the percentage volume of packed erythrocytes. Hemoglobin of the tail blood was measured with cuvette haemoglobin kit (HemoCue AB, Baumann Medical AG, Zurich, Switzerland).

### Histochemistry

Secondary antibodies for peroxidase/DAB stainings were from Vector Laboratories (Burlingame, Calif., USA) and for immunofluorescent stainings from Jackson (Milan Analytic, Fribourg, Switzerland). Mouse monoclonal antibody 6E10 (Signet, Dedham, USA) 1:400; rat monoclonal antibody against CD31 (BD Biosciences, Basel, Switzerland) 1:100; rabbit polyclonal antibodies against GFAP (Sigma, Buchs, Switzerland) 1:400 and claudin-5 (Invitrogen, Basel) 1:100 were used for immunohistochemistry. Ferric iron was detected by Perls staining according to a standard protocol.

### Immunohistochemistry

Primary antibodies and dilutions used for immunohistochemical staining were: mouse monoclonal antibody 6E10 (Signet, Dedham, USA) 1:400; rabbit polyclonal antibodies against C-terminal amyloid precursor protein (APP) 1:500, and anti-GFAP 1:400 (Sigma, Buchs, Switzerland). Secondary antibodies used for peroxidase/DAB stainings were from Vector Laboratories (Burlingame, Calif., USA) and for immunofluorescent stainings from Jackson (Milan Analytic, Fribourg, Switzerland).

### Protein Extracts and Western Blotting

Each left hemisphere was homogenized with a glass teflon homogenizer in 15 volume of RIPA buffer (0.5% sodium deoxycholate, 0.1% SDS, 150 mM NaCl, 50 mM TrisCl (pH 8.0), 5 mM EDTA, 1 mM Na3VO4, 1 mM NaF, 1× protease inhibitor cocktail (Sigma) and 1 mM AEBSF). After 40 min centrifugation at 100,000 g at 4° C., supernatant was collected as RIPA fraction. The pellet was suspended in 0.7 ml RIPA buffer and recollected after 30 min centrifugation at 100,000 g 4° C. The pellet was then suspended in 0.5 ml RIPA, 2 mM EDTA and 2% SDS. Supernatant was collected as SDS fraction after 40 min centrifugation at 100,000 g 8° C. The pellet was then resolved in 75 μl 70% formic acid. The suspension was neutralized with 1.5 ml 1 M Tris (pH 11), and centrifuged at 20,000 g 4° C. for 30 min. The supernatant was then collected as FA fraction. Extracts of each fraction were separated by 10-20% tricine SDS-PAGE, blotted onto nitrocellulose membrane, and boiled for 5 min in PBS. Primary antibodies including 6E10 (1:300), CD31 (1:50), rabbit polyclonal antibodies against claudin-5 (1:100), C-terminal APP (Sigma, Basel, Switzerland, 1:2,000) and PKAα cat (Santa Cruz, Basel, Switzerland, 1:100), mouse monoclonal antibodies against β-actin (Abcam, Cambridge, UK, 1:3,000) and GAPDH (Biodesign, Fribourg, Switzerland, 1:3000). Target proteins were visualized by peroxidase-conjugated secondary antibodies and ECL reactions (Amersham Biosciences, Otelfingen, Switzerland).

### ELISAs

hAmyloid β40 ELISA kit (The Genetics Company, Zurich, Switzerland) was used for quantifying the level of Aβ40, and INNOTEST® (Innogenetics, Heiden, Germany) for Aβ42. Brain extracts and sera were diluted according to previous titration studies.

### Microvessel Extraction

Cerebral microvessels were isolated based on the established method (Banks, 1999) with slight modification. Briefly, brains without cerebellum were freed from visible blood vessels and meninges in ice-cold HBSS, then minced with a scalpel blade into approximately 1 mm3 in 5 ml ice-cold DMEM:F-12 (GIBCO, 32500-035, Basel, Switzerland) containing 1% dextran. The cut-up tissue was then homogenized in a 7-ml Dounce homogenizer (30 strokes with the larger clearance pestle followed by 25 strokes with the smaller clearance pestle) in ice-cold DMEM-F-12. The resulting homogenate was centrifuged at 200 g 4° C. for 5 min. The pellet was re-suspended in 15 ml 20% dextran-DMEM-F12 and centrifuged at 4,500 g 4° C. for 15 min. The pellet was re-suspended in 1% dextran DMEM-F-12 and passed through 40 μm mesh membrane. Microvessels were collected by washing the membrane with 25 ml HBSS followed by centrifugation at 1000 g 4° C. for 5 min. Microvessels were than fixed in 4% PFA at room temperature for 10 min and stored in PBS (pH 7.4) containing 0.05% NaN3 at 4° C. before use.

### Cell Culture and Treatments

Murine endothelial cell lines, bEnd3 and bEnd5 cells were immortalized brain endothelial cell lines established from mouse brain microvessels using the polyoma virus middle T-antigen and may be obtained from commercial cell banks such as the European Collection of Animal Cell Cultures (ECACC) or the American Tissue Culture Collection (ATCC); see also Williams et al., Cell 57 (1989), 1053±1063. These cells were cultured on Petri dish in DMEM (4.5 g/L glucose) containing 10% FCS (heat inactivated), 4 mM L-Glutamine, 1× MEM non-essential amino acids, 1 mM sodium pyruvate, 100 units/ml penicillin, 100 μg/ml streptomycin and 50 μM beta-mecaptoethanol at a density of 5×10⁴/cm² at 37° C. with 5% CO₂ for two weeks with twice a week medium change. Cells on Petri dish were then treated with 10 82 M freshly prepared synthetic Aβ42 (Bachem, Basel, Switzerland) or 1 UI/ml rHuEPO or both in PBS for 24 hrs. the control culture was treated with equal volume of PBS. The cells were then washed twice with PBS and immediately frozen on dry ice. Cells were then scraped off in 200 μl RIPA on ice. The cell suspension was collected and ultrasonificated for 30 sec before subjected to centrifugation at 14,000 rpm 4° C. for 30 min. Supernatants were collected and stored at −80° C. in aliquots before use. For immunocytochemistry study, cells were cultured in multi-chamber culture slides.

SweAPP293 cells, i.e. HEK 293 cells expressing beta-amyloid precursor protein with the Swedish double mutation (see, e.g., the 20E2 cell line which is a Swedish mutant APP695 stable HEK cell line (Qing et al., FASEB J. 18 (2004), 1571-1573) were cultured in DMEM containing 10% FCS, 100 units/ml penicillin, 100 ug/ml streptomycin, on 6 cm Petri dish with a density of 2.5×104/cm2 at 37° C. with 5% CO2. Eight hours later, culture medium was refreshed with rHuEPO at various concentrations (0, 0.0001, 0.01, 0.1, 1 and 10 UI/ml) or together with 1 μM DAPT (γ-secretase inhibitor IX, Calbiochem). Conditioned media were collected 24 hrs and 46 hrs later, followed by centrifugation at 4° C. 2500 rpm for 10 min. Cells were first frozen in dry ice and then scraped in 200 μl RIPA buffer. The cell suspensions were then subjected to ultrasonification for 30 sec. Total cellular protein extracts were collected after 30 smin centrifugation at 4° C. 14,000 rpm. Conditioned media and cellular protein extracts were stored at −80° C. before use.

### Human Brain Sample Preparation

Frozen Temporal cortexes from AD patients and healthy controls (post mortem delay <3.5 hrs) were homogenized in RIPA buffer. Supernatants were collected after 40 min centrifugation at 4° C., 22000×g and protein content was quantified by BioRad DC-assay. Supernatants and pellets were stored at −80° C. before use.

### Statistics

Data were analyzed with SPSS version 11.5. One-way ANOVA was used to assess differences among treated groups followed by LSD multiple comparisons with a significant level at 0.05. Student t-test was used to assess differences between two groups with a significant level at 0.05. Correlation between two variables was tested by Pearson or Spearman's rho correlation, and was considered significant when P<0.01 in Pearson and P<0.05 in Spearman's rho test.

### Example 1

### rHuEPO Reduces the Number of Aβ Plaques and Aβ Plaque-associated Astrocytosis in the Brain

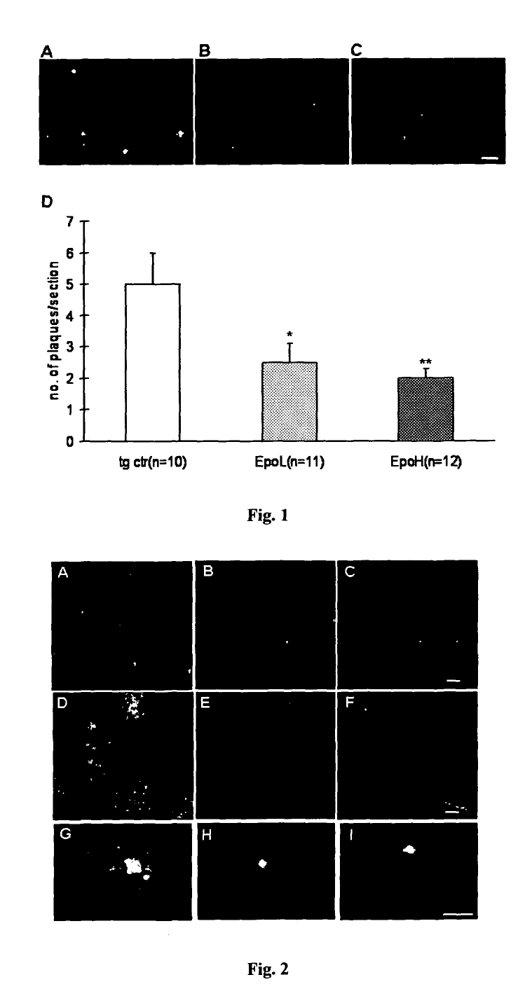

At eight months of age, arcAβ mice had already developed marked Aβ deposits in the brain parenchyma and leptomeningeal and parenchymal blood vessels, which were revealed by 6E10 immunofluorescence staining. Most of the 6E10-positive Aβ deposits were confirmed by thioflavin-S staining as neuritic plaques (FIGS. 1A, B and C). Thioflavin-S plaques appeared predominantly in the cortex (5.0±1.09/section), but rarely in the hippocampus. The number of plaques was reduced by more than 40% in EpoL and EpoH (2.6±0.6/section and 2.0±0.3/section respectively; P<0.05 and 0.01, LSD, FIG. 1D). Astrocytosis in response to Aβ accumulation in brain parenchyma was already prominent in eight-month-old tg ctr (FIG. 2D). In contrast, Aβ plaque-associated astrocytosis was markedly reduced in EpoL (FIG. 2E) and EpoH (FIG. 2F), as determined by the number of GFAP-positive cells and the fluorescence intensity surrounded each 6E10-positive Aβ plaque (FIGS. 2A, B and C). This was unlikely due to a reduced plaque size, because surround plaques of similar size, marked reductions of astrocytosis were detected in EpoL (FIG. 2H) and EpoH (FIG. 2I). Thus, chronic rHuEPO treatments reduced the number of Aβ plaques and associated astrocytosis in the brain of arcAβ mice.

### Example 2

### rHuEPO Reduces CAA and Maintains the Close Contact Between Astrocytes and Blood Vessel

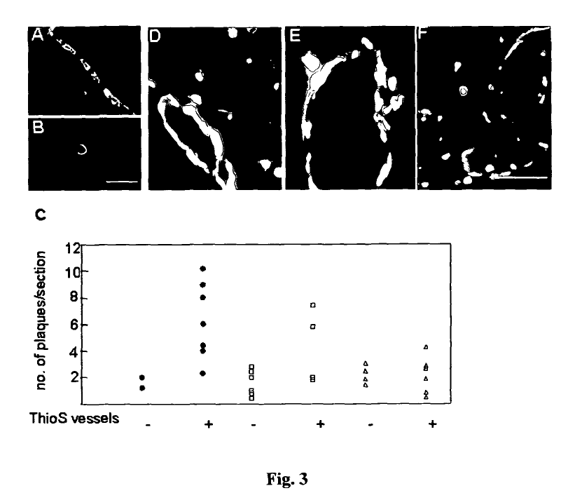

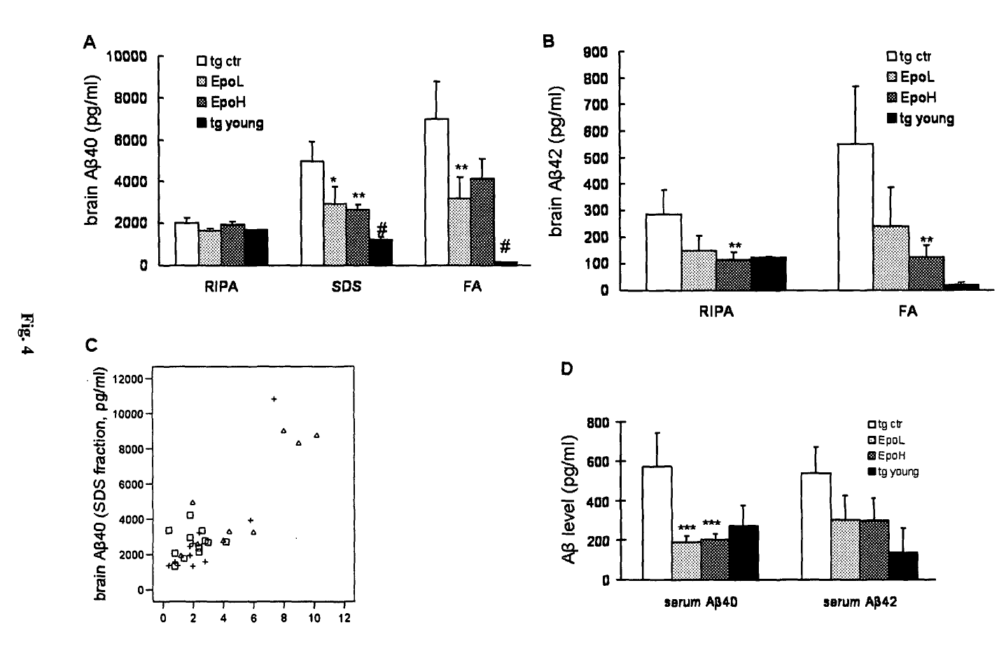

Thioflavin-S staining also revealed significant CAA in eight-month-old arcAβ mice, both in the leptomeninges and the cortex (FIGS. 3A and B). The appearance of thioflavin-S stained vessels positively correlated to the number of thioflavin-S plaques in the cortex (P<0.05, r=0.725, Spearman's rho correlation coefficient, FIG. 3C). However, thioflavin-S stained vessels were less prominent in EpoL and EpoH (5 of 11 and 7 of 12 respectively). Interestingly, there was no association between the number of thioflavin-S plaques and the appearance of thioflavine-S vessels in EpoL and EpoH mice (P=0.253 and 0.647 respectively, FIG. 3C). However, Perls staining of ferric iron did not detect any microhemorrhage in brains of all four groups (unpublished data). This indicates microhemorrhage occurred in arcAβ mice later than did CAA. In addition, despite being highly GFAP-reactive, astrocytes mostly detached from Aβ-laden blood vessels (FIG. 3D). In contrast, GFAP-positive astrocytes remained in close contact with Aβ-laden blood vessels in both EpoL (FIG. 3E) and EpoH (FIG. 3F). Thus, chronic rHuEPO treatments prevented the disassociation of astrocytes from the blood vessels and reduced CAA in arcAβ mice.

### Example 3

### rHuEPO Lowers Brain and Serum Aβ Levels

Brain Aβ in RIPA, SDS and FA fractions were quantified with ELISA. Compared with four-month-old arcAβ mice which had no detectable Aβ plaques in the brain, eight-month-old tg ctr mice had only a slight increase in Aβ40 in RIPA fraction (P=0.210), but a four-fold increase in SDS fraction and a 40-fold increase in FA fraction (P<0.01, FIG. 4A). Chronic rHuEPO treatments reduced the Aβ40 level only slightly in RIPA fraction (P=0.211, FIG. 4A), but dramatically in less soluble fractions by more then 40%. The reduction of Aβ40 was significant in SDS fraction in EpoL, as well as in FA fraction in EpoH (P<0.05, FIG. 4A). The levels of brain Aβ42 in rHuEPO treated mice reduced by a similar degree and the reductions were significant in RIPA and FA fraction of EpoH (P<0.05, FIG. 4B). Further analyses showed that the number of thioflavin-S plaques was positively associated with brain Aβ levels in all RIPA-insoluble fractions (P<0.001, Pearson correlation), and the association was strongest with Aβ40 level in SDS fraction (r=0.791, P<0.001, FIG. 4C). In addition, a more than 40% decrease in serum Aβ40 was also seen in EpoL and EpoH (P<0.01, FIG. 4D). Thus, chronic rHuEPO treatments significantly reduced brain Aβ levels in RIPA-insoluble fractions and serum Aβ40 in arcAβ mice.

### Example 4

### rHuEPO Activates Non-amyloidogenic Processing of APP

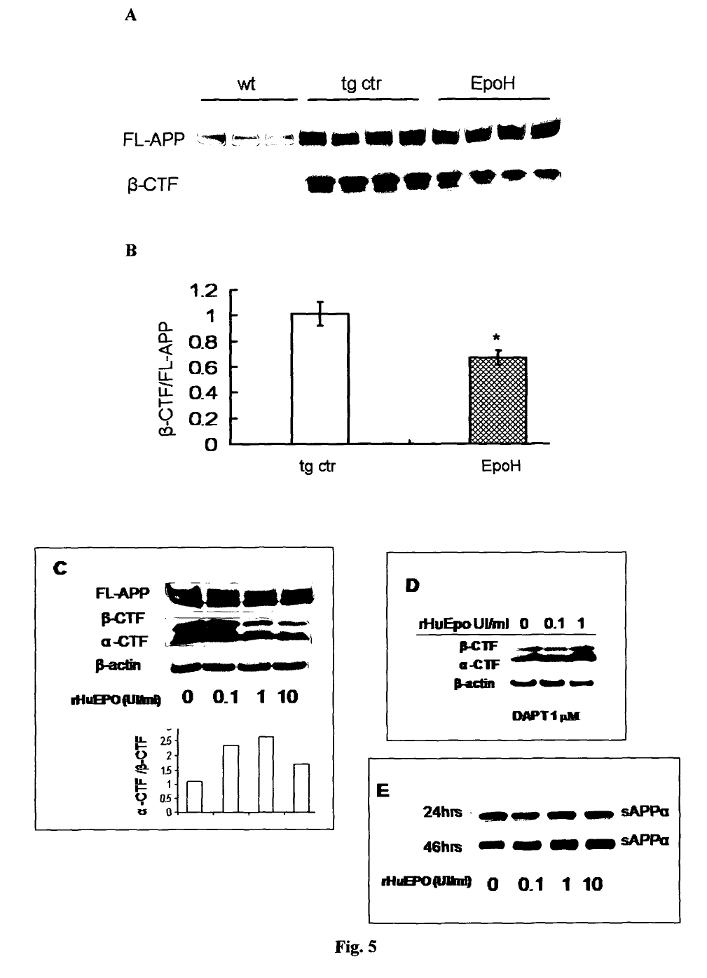

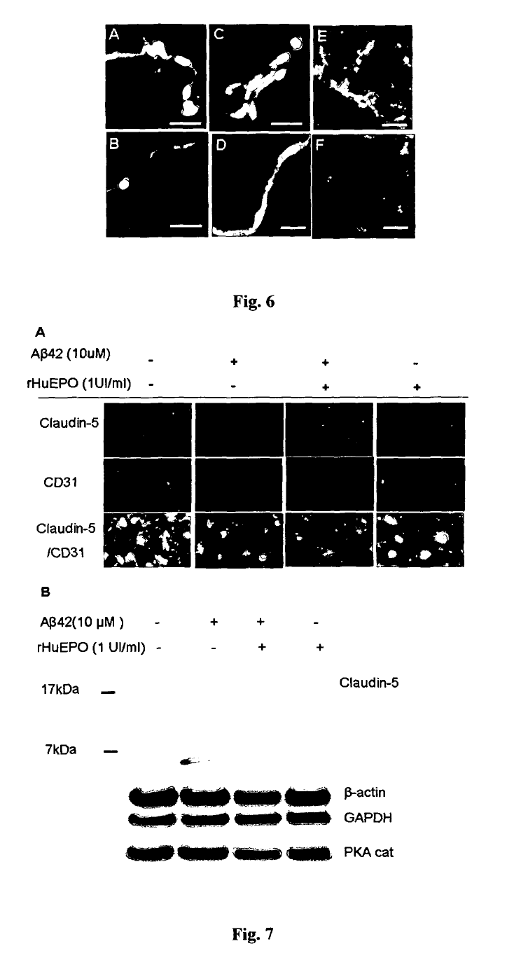

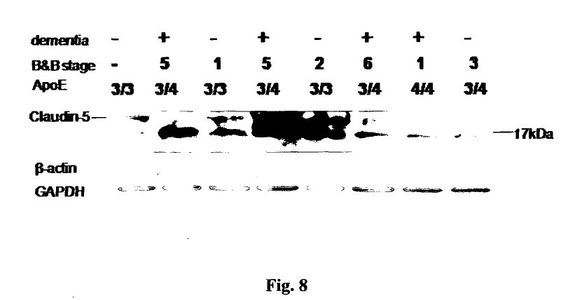

The β-cleavage of APP by β-secretase BACE1 is the first step in amyloidogenic processing of APP and thus amyloidogenesis. Thus, the level of C-terminal fragment of β-cleavage, β-CTF, indirectly reflects the degree of Aβ production in mice. The levels of β-CTF in tg ctr mice were apparently higher than in EpoH mice as revealed by Western blots (FIG. 5). In order to rule out the possibility that this was due to a difference in APP synthesis, the densitometory measurement of β-CTF was normalized to that of the full length APP (FL-APP) in each individual mouse on the same Western blot. The ratio of β-CTF to FL-APP was significantly reduced by 34% in rHuEPO treated mice (EpoH) compared with tg ctr (n=7, student-test, p<0.01). These results could be confirmed with SweAPP293 cells overexpressing human APP695 containing the Swedish mutation which were subjected to rHuEpo. The levels of α-CTF and β-CTF in SweAPP cells were approximately equal on Western blot (C). rHuEPO at various concentrations markedly increased α-CTF/β-CTF, which peaked at 1 UI/ml with α-CTF/β-CTF at 2.6. However, DAPT, an established γ-cleavage inhibitor, failed to block the increase in α-CTF/β-CTF by rHuEPO (D). In addition, the extracellular fragment of α-cleavage, sAPPα, was also increased in the conditioned media from rHuEPO treated cells (E). Thus, rHuEPO favored nonamyloidogenic processing of APP in arcAβ mice and in SweAPP293 cells.

### Example 5

### rHuEPO Prevents Aβ Toxicity on Microvessel Endothelial Cells

Apart from CAA, arcAβ mice had also severe damage of brain microvessel integrity. Claudin-5 is a major component of tight junction in brain microvessels. Western blot indicated a trend of reduction in brain claudin-5 in arcAβ mice, and the reduction was partially recovered in both rHuEPO treated groups. Since claudin-5 is enriched in brain microvessels, brain microvessels was further extracted from wt, tg ctr and rHuEPO treated mice. In wt, microvessels were composed of endothelial cells with a distance interval of about 40 μm between two adjacent nuclei. Claudin-5 was evenly distributed along the vessel wall, where Aβ was absent (FIG. 6A). However, in the vessel wall of Aβ-laden microvessels isolated from arcAβ mice, the inter-nuclear distance between adjacent endothelial cells was often larger (FIG. 6D), and in some cases, a loss of endothelial cells was apparent (FIG. 6B). However, the distribution of platelet endothelial adhesion molecule-1 (PECAM-1/CD31), another endothelial marker, was unaffected even in Aβ-laden microvessels (FIG. 6D). Microvessels were fully enveloped by astrocytes in wt (FIG. 6E), but often not in tg ctr, where the distribution of claudin-5 was also disrupted (FIG. 6F). Interestingly, rHuEPO partially restored the normal distribution of claudin-5 in Aβ-laden microvessels (FIG. 6C).

The toxic effect of Aβ towards claudin-5 was further studied in bEnd5 cells, an endothelial cell line established from mouse brain microvessels. Claudin-5 was mainly expressed in the cell membrane in bEnd5 cells (FIG. 7A). When these cells were cultured in freshly prepared 10 μM Aβ42 for 24 hrs, claudin-5 disassociated from the cell membrane and accumulated in the cytoplasm. Addition of 1 UI/ml rHuEPO together with 10 82 M Aβ42 preserved membrane-bound claudin-5, although cytoplasmic claudin-5 still existed. However, 1 UI/ml rHuEPO alone did not change the normal distribution pattern of claudin-5. Despite the complete disappearance of claudin-5 from cell membrane by Aβ42, there was no significant reduction in the protein level of claudin-5 (FIG. 7B). Furthermore, Aβ42 induced a significant increase in the level of C-terminal fragments of claudin-5, which were hardly detectable in control and rHuEPO treated cells, whereas addition of 1 UI/ml rHuEPO markedly reduced these C-terminal fragments (FIG. 7B). Together, these data suggest that claudin-5 was sensitive to Aβ, both in vivo and in vitro. rHuEPO prevented Aβ toxicity toward claudin-5 both in mice and in bEnd5 cells.

### Example 6

### Chronic rHuEPO Treatment does not Affect General Physiological Conditions in arcAβ Mice

To assess whether weekly rHuEPO treatment was associated with increased erythropoiesis in arcAβ mice, the levels of hemoglobin and hematocrit were measured in all four groups. Both parameters remained unchanges in the EpoL and EpoH treatment groups (p=0.387 and p=0.465, respectively). The general physiological conditions were monitored by mini-neurological examination, which measures parameters of coat appearance, body weight, body temperature, secretory signs, body posture and basic reflexes including eye blink, pupillary, flexion and righting reflexes. Muscular strength indicated as grip strength was measured with a spring scale. No significant differences were observed between the tg ctr and rHuEPO treated mice which appeared normal and and comparable to the saline treated wt littermates. Thus, weekly ip injection of 600 UI or 60 UI/kg rHuEPO did not significantly increase the hematocrit nor exert any obvious adverse effects.

### Example 7

### Claudin-5 is a Marker of Brain Vasculature Damage in Alzheimer's Disease

Western blot reveals the level of claudin-5 in the temporal cortexes from demented patients and healthy controls. Subjects only with a clinical diagnose of dementia were labeled as +, clinically non-demented subjects as −. The severity of neurofibrillary tangles was indicated by Braak and Braak Stage (B&B stage). In addition, the apolipoprotein E genotype of each subject was also indicated. In all demented patients, the full length form of claudin-5 was absent while a smaller band of 16 kDa was detected as the dominant form; see FIG. 8. The difference in the level of full length claudin-5 and the appearance of low molecular weight forms between demented patients and healthy control subjects, strongly suggests that claudin-5 is a prominent marker of brain vasculature damage in Alzheimer's disease.

The present invention is the first disclosure to demonstrate a beneficial role of rHuEPO in AD model mice, although rHuEPO has been successfully used for preventing severe brain damage and memory impairment caused by hypoxic-ischemia (Kumral et al., 2004), glutamate excitotoxicity (Miu et al., 2004) and traumatic brain injury (Lu et al., 2005) in rodents. The arcAβ mice have two major pathophysiological features:
(1) Aβ accumulation both in the brain parenchyma and blood vessels;
(2) compromised brain microvessel integrity.

Due to the use of this mouse model it could be shown that rHuEPO reduced the brain Aβ levels and improved the brain microvessel integrity. In summary, it is disclosed for the first time that systematic rHuEPO treatment lowered brain Aβ levels and improved the brain microvessel integrity in AD mice, thus open up a novel approach for the treatment of AD. In addition, a novel biomarker for AD, claudin-5 and its 16 kDa variant form could be identified,

### REFERENCES

Banks W A (1999) Characterization of interleukin-1alpha binding to mouse brain endothelial cells. J Pharmacol Exp Ther 291:665-670.
Brines M, Cerami A (2005) Emerging biological roles for erythropoietin in the nervous system. Nat Rev Neurosci 6:484-494.
Brines M, Grasso G, Fiordaliso F, Sfacteria A, Ghezzi P, Fratelli M, Latini R, Xie Q W, Smart J, Su-Rick C J, Pobre E, Diaz D, Gomez D, Hand C, Coleman T, Cerami A (2004) Erythropoietin mediates tissue protection through an erythropoietin and common beta-subunit heteroreceptor. Proc Natl Acad Sci USA 101:14907-14912.
Brines M L, Ghezzi P, Keenan S, Agnello D, de Lanerolle N C, Cerami C, Itri L M, Cerami A (2000) Erythropoietin crosses the blood-brain barrier to protect against experimental brain injury. Proc Natl Acad Sci USA 97:10526-10531.
Buemi M, Cavallaro E, Floccari F, Sturiale A, Aloisi C, Trimarchi M, Grasso G, Corica F, Frisina N (2002) Erythropoietin and the brain: from neurodevelopment to neuroprotection. Clin Sci (Lond) 103:275-282.
Carmichael S T (2006) Cellular and molecular mechanisms of neural repair after stroke: making waves. Ann Neurol 59:735-742.
Chong Z Z, Li F, Maiese K (2005) Erythropoietin requires NF-kappaB and its nuclear translocation to prevent early and late apoptotic neuronal injury during beta-amyloid toxicity. Curr Neurovasc Res 2:387-399.
Egrie J C, Dwyer E, Browne J K, Hitz A, Lykos M A (2003) Darbepoetin alfa has a longer circulating half-life and greater in vivo potency than recombinant human erythropoietin. Exp Hematol 31:290-299.
Ehrenreich H, Hasselblatt M, Dembowski C, Cepek L, Lewczuk P, Stiefel M, Rustenbeck H H, Breiter N, Jacob S, Knerlich F, Bohn M, Poser W, Ruther E, Kochen M, Gefeller O, Gleiter C, Wessel T C, De Ryck M, Itri L, Prange H, Cerami A, Brines M, Siren A L (2002) Erythropoietin therapy for acute stroke is both safe and beneficial. Mol Med 8:495-505.
Gurney K J, Estrada E Y, Rosenberg G A (2006) Blood-brain barrier disruption by stromelysin-1 facilitates neutrophil infiltration in neuroinflammation. Neurobiol Dis 23:87-96.
Hardy J, Selkoe D J (2002) The amyloid hypothesis of Alzheimer's disease: progress and problems on the road to therapeutics. Science 297:353-356.
Iadecola C (2004) Neurovascular regulation in the normal brain and in Alzheimer's disease. Nat Rev Neurosci 5:347-360.
Jaquet K, Krause K, Tawakol-Khodai M, Geidel S, Kuck K H (2002) Erythropoietin and VEGF exhibit equal angiogenic potential. Microvasc Res 64:326-333.
Koto T, Takubo K, Ishida S, Shinoda H, Inoue M, Tsubota K, Okada Y, Ikeda E (2007) Hypoxia disrupts the barrier function of neural blood vessels through changes in the expression of claudin-5 in endothelial cells. Am J Pathol 170:1389-1397.
Kumar-Singh S, Pirici D, McGowan E, Serneels S, Ceuterick C, Hardy J, Duff K, Dickson D, Van Broeckhoven C (2005) Dense-core plaques in Tg2576 and PSAPP mouse models of Alzheimer's disease are centered on vessel walls. Am J Pathol 167:527-543.
Kumral A, Uysal N, Tugyan K, Sonmez A, Yilmaz O, Gokmen N, Kiray M, Genc S, Duman N, Koroglu T F, Ozkan H, Genc K (2004) Erythropoietin improves long-term spatial memory deficits and brain injury following neonatal hypoxia-ischemia in rats. Behav Brain Res 153:77-86.
Li Y, Lu Z, Keogh C L, Yu S P, Wei L (2007) Erythropoietin-induced neurovascular protection, angiogenesis, and cerebral blood flow restoration after focal ischemia in mice. J Cereb Blood Flow Metab 27:1043-1054.
Lu D, Mahmood A, Qu C, Goussev A, Schallert T, Chopp M (2005) Erythropoietin enhances neurogenesis and restores spatial memory in rats after traumatic brain injury. J Neurotrauma 22:1011-1017.
Marco S, Skaper S D (2006) Amyloid beta-peptide1-42 alters tight junction protein distribution and expression in brain microvessel endothelial cells. Neurosci Lett 401:219-224.
Miu A C, Olteanu A I, Chis I, Heilman R M (2004) Have no fear, erythropoietin is here: erythropoietin protects fear conditioning performances after functional inactivation of the amygdala. Behav Brain Res 155:223-229.
Nadam J, Navarro F, Sanchez P, Moulin C, Georges B, Laglaine A, Pequignot J M, Morales A, Ryvlin P, Bezin L (2007) Neuroprotective effects of erythropoietin in the rat hippocampus after pilocarpine-induced status epilepticus. Neurobiol Dis 25:412-426.
Ogunshola O O, Djonov V, Staudt R, Vogel J, Gassmann M (2006) Chronic excessive erythrocytosis induces endothelial activation and damage in mouse brain. Am J Physiol Regul Integr Comp Physiol 290:R678-684.
Park L, Anrather J, Forster C, Kazama K, Carlson G A, Iadecola C (2004) Abeta-induced vascular oxidative stress and attenuation of functional hyperemia in mouse somatosensory cortex. J Cereb Blood Flow Metab 24:334-342.
Rifkind J M, Abugo O O, Peddada R R, Patel N, Speer D, Balagopalakrishna C, Danon D, Ingram D K, Spangler E L (1999) Maze learning impairment is associated with stress hemopoiesis induced by chronic treatment of aged rats with human recombinant erythropoietin. Life Sci 64:237-247.
Soma T, Chiba H, Kato-Mori Y, Wada T, Yamashita T, Kojima T, Sawada N (2004) Thr(207) of claudin-5 is involved in size-selective loosening of the endothelial barrier by cyclic AMP. Exp Cell Res 300:202-212.
Sun X, He G, Qing H, Zhou W, Dobie F, Cai F, Staufenbiel M, Huang L E, Song W (2006) Hypoxia facilitates Alzheimer's disease pathogenesis by up-regulating BACE1 gene expression. Proc Natl Acad Sci USA 103:18727-18732.
Tsai P T, Ohab J J, Kertesz N, Groszer M, Matter C, Gao J, Liu X, Wu H, Carmichael S T (2006) A critical role of erythropoietin receptor in neurogenesis and post-stroke recovery. J Neurosci 26:1269-1274.
Villa P, Bigini P, Mennini T, Agnello D, Laragione T, Cagnotto A, Viviani B, Marinovich M, Cerami A, Coleman T R, Brines M, Ghezzi P (2003) Erythropoietin selectively attenuates cytokine production and inflammation in cerebral ischemia by targeting neuronal apoptosis. J Exp Med 198:971-975.
Wang L, Zhang Z G, Zhang R L, Jiao Z X, Wang Y, Pourabdollah-Nejad D S, LeTourneau Y, Gregg S R, Chopp M (2006) Neurogenin 1 mediates erythropoietin enhanced differentiation of adult neural progenitor cells. J Cereb Blood Flow Metab 26:556-564.
Willis C L, Leach L, Clarke G J, Nolan C C, Ray D E (2004a) Reversible disruption of tight junction complexes in the rat blood-brain barrier, following transitory focal astrocyte loss. Glia 48:1-13.
Willis C L, Nolan C C, Reith S N, Lister T, Prior M J, Guerin C J, Mavroudis G, Ray D E (2004b) Focal astrocyte loss is followed by microvascular damage, with subsequent repair of the blood-brain barrier in the apparent absence of direct astrocytic contact. Glia 45:325-337.
Yamaji R, Okada T, Moriya M, Naito M, Tsuruo T, Miyatake K, Nakano Y (1996) Brain capillary endothelial cells express two forms of erythropoietin receptor mRNA. Eur J Biochem 239:494-500.

1. A pharmaceutical composition comprising erythropoietin (EPO) or a biologically active fragment or analog thereof for the treatment, amelioration, or prevention of a neurological disorder or amyloidosis in a subject.

2. The pharmaceutical composition of claim 1, wherein said disorder involves damaged microvessel endothelium in the brain.

3. The pharmaceutical composition of claim 2, wherein the damaged microvessel endothelium is characterized by cell membrane disassociation of claudin-5 and/or its reduced protein level.

4. The pharmaceutical composition of claim 1, wherein the disorder is associated with amyloidosis.

5. The pharmaceutical composition of claim 1, wherein the disorder is associated with amyloid β (Aβ) pathology.

6. The pharmaceutical composition of claim 1, wherein the disorder results from amyloid precursor protein (APP) amyloidogenic processing.

7. The pharmaceutical composition of claim 1, wherein said neurological disorder is Alzheimer's disease.

8. The pharmaceutical composition of claim 1, wherein EPO or an active fragment or analog thereof is designed to be applied exogenously to or expressed in a target cell.

9. The pharmaceutical composition of claim 8, wherein said target cell is a capillary endothelial cell in the brain.

10. The pharmaceutical composition of claim 1, wherein said EPO is human EPO or a biologically active fragment thereof.

11. The pharmaceutical composition of claim 1, wherein said EPO or active fragment or analog thereof is hyper-glycosylated compared to native human EPO.

12. The pharmaceutical composition of claim 1, wherein said EPO or active fragment or analog thereof is Darbepoietin.

13. The pharmaceutical composition of claim 1, which is designed to be administered systemically.

14. The pharmaceutical composition of claim 1, which is designed to be administered in a therapeutic effective amount without significantly increasing the hemoglobin level in the subject.

15. The pharmaceutical composition of claim 1, which is designed to be administered in a therapeutic effective amount without significantly increasing the hematocrit of the subject.

16. The pharmaceutical composition of claim 1, which is designed to be administered at a dose of between about 1 UI/kg and below 1000 UI/kg.

17. The pharmaceutical composition of claim 1, which is designed to be administered at a dose of at most 100 UI/kg.

18. The pharmaceutical composition of claim 1, which is designed to be administered weekly.

19. The pharmaceutical composition of claim 1, further comprising an anti-Aβ antibody or equivalent Aβ binding molecule or designed to be administered in conjunction with a pharmaceutical composition comprising such anti-Aβ antibody or equivalent Aβ binding molecule.

20. The pharmaceutical composition of claim 1, comprising an anti-Aβ antibody or equivalent Aβ binding molecule or a combined preparation thereof for simultaneous, separate or sequential use in Alzheimer therapy.

21. A method for the treatment, amelioration, or prevention of a neurological disorder in a subject, comprising administering to said subject a pharmaceutical composition according to claim 1, wherein the EPO or an active fragment or analog thereof is administered at a dose of at most 1000 UI/kg, thereby preventing or reducing the severity of the neurological disorder.

22. A method for the treatment, amelioration, or prevention of damaged microvessel endothelium in the brain of a subject characterized by cell membrane disassociation of claudin-5 and/or its reduced protein level, comprising administering to said subject a pharmaceutical composition according to claim 1, wherein the EPO or an active fragment or analog thereof is present in an amount sufficient to treat, ameliorate, or prevent damaged microvessel endothelium in the brain, thereby preventing or reducing the severity of the damage.

23. A method for assessing Alzheimer's disease in vitro comprising measuring in a body fluid sample the level of caudin-5 or a variant thereof, wherein a decreased level of claudin-5 and/or increased level of said variant thereof as compared to a reference value of sample from a healthy subject is indicative that said individual suffers from or is at risk to suffer from Alzheimer's disease.

24. An in vitro method for monitoring the progression of the Alzheimer's disease comprising measuring in a body fluid sample the level of caudin-5 or a variant thereof, wherein a decreased level of claudin-5 and/or increased level of said variant, compared with an earlier measurement of the level of claudin-5 or said variant thereof is indicative for the progression of Alzheimer's disease.

25. The method according to claim 24, wherein the body fluid is cerebrospinal fluid or blood.

26. (canceled)

27. (canceled)

28. A kit for use in a method of claim 24, said kit comprising a means or an agent for measuring claudin-5 or variant thereof.

29. (canceled)

30. (canceled)
